(19) United States
(12) Patent Application Publication
(10) Pub. No.: US 2014/0357919 A1
(43) Pub. Date: Dec. 4, 2014

(54) PROCESS FOR THE OXIDATIVE REGENERATION OF A DEACTIVATED CATALYST AND AN APPARATUS THEREFOR

(71) Applicants: SHELL INTERNATIONALE RESEARCH MAATSCHAPPIJ B.V., HR THE HAGUE (NL); SHELL OIL COMPANY, HOUSTON, TX (US)

(72) Inventors: Sivakumar Sadasivan Vijayakumari, Amstedam (NL); Jeroen Van Westrenen, Amsterdam (NL)

(21) Appl. No.: 14/360,855

(22) PCT Filed: Nov. 28, 2012
(86) PCT No.: PCT/EP2012/073780

§ 371 Date: May 27, 2014

(51) Int. Cl. B01J 29/90; C07C 1/22; C07C 4/06

(52) U.S. Cl. CPC B01J 29/90; C07C 4/06; C07C 1/22; C07C 2523/02; C07C 2523/04
USPC 585/638; 502/33; 502/30; 502/34; 502/38; 585/651; 585/653; 422/187

(57) ABSTRACT

The present invention relates to a process for the oxidative regeneration of a deactivated catalyst comprising molecular sieve to provide a regenerated molecular sieve catalyst, wherein said deactivated catalyst is from one or both of an oxygenate to olefin process and a olefin cracking process, said regeneration process comprising at least the steps of providing a regeneration gas stream comprising oxidant; treating the regeneration gas stream with a liquid adsorbent stream comprising an ethylene glycol in a contaminant absorption zone to remove at least a part of one or more of any water, any alkali metal ion and any alkaline earth metal ion present in the regeneration gas stream to provide a treated regeneration gas stream comprising oxidant; regenerating a deactivated catalyst comprising molecular sieve with the treated regeneration gas stream to provide a regenerated catalyst comprising regenerated molecular sieve.

### FIELD OF THE INVENTION

The present invention relates to a process for the oxidative regeneration of a deactivated catalyst comprising molecular sieve, such as zeolite, specifically deactivated catalyst from one or both of an oxygenate to olefin (OTO) process and an olefin cracking process (OCP) process, an apparatus therefor and a process for the preparation of olefinic product incorporating such a regeneration process.

### BACKGROUND OF THE INVENTION

Conventionally, ethylene and propylene are produced via steam cracking of paraffinic feedstocks including ethane, propane, naphtha and hydrowax. An alternative route to ethylene and propylene is an oxygenate-to-olefin (OTO) process. Interest in OTO processes for producing ethylene and propylene is growing in view of the increasing availability of natural gas. Methane in the natural gas can be converted into for instance methanol or dimethyl ether (DME), both of which are suitable feedstocks for an OTO process.

In an OTO process, an oxygenate such as methanol is provided to a reaction zone of a reactor comprising a suitable conversion catalyst whereby the oxygenate is converted to ethylene and propylene. In addition to the desired ethylene and propylene, a substantial part of the oxygenate is converted to higher hydrocarbons including C4+ olefins and paraffins. The effluent from the reactor comprising the olefins, any unreacted oxygenates such as alcohols or ethers, particularly methanol and dimethyl ether and other reaction products such as water may then be treated to provide separate component streams. Unreacted oxygenates and water can be separated from the reaction effluent, for instance by contacting with a cooled aqueous stream in a quench zone. In order to increase the ethylene and propylene yield of the process, the C4+ olefins may be recycled to the reaction zone or alternatively further cracked in a dedicated olefin cracking zone to produce further ethylene and propylene.

International patent application no. PCT/US2005/025666 (WO 2006/023189) discloses a process for regenerating a molecular sieve catalyst. Molecular sieve catalyst used for converting oxygenates to olefins requires periodic regeneration in order to maintain catalyst activity. The regeneration can burn off carbonaceous deposits formed on the catalyst during oxygenate conversion and can be carried out in the presence of oxygen.

WO 2006/023189 discloses that the molecular sieve catalysts were found to be sensitive to various contaminants. In particular, contaminants such as sodium chloride, which can be present in the air used in regeneration, were found to cause serious damage to the molecular sieve catalysts. For more valuable catalyst systems, such as those used in the methanol to olefins process, it is said to be economically advantageous to remove airborne salts as well as humidity from the regeneration air. The process of WO 2006/023189 employs a cooler and a scrubber to wash the salts from the air. The condensed water formed in the cooler can be used in the scrubber to wash out much of the air-borne salt. The use of water in the scrubber can result in an incidental increase in humidity, such that supplemental driers or demisters can be added.

One problem with the process of WO 2006/023189 is that it requires the regeneration air to be cooled before scrubbing. The scrubbed regeneration air must then be reheated prior to regeneration. In addition, the use of an aqueous scrubber to wash out salt increases the humidity of the regeneration air produced by the scrubber through entrained droplets. Water molecules may deactivate the catalytic sites of molecular sieve catalysts permanently. Thus, additional devices such as the use of driers or demisters may be required to reduce the humidity of the scrubbed regeneration air.

### SUMMARY OF THE INVENTION

The present invention provides an improved process of, and apparatus for, regenerating a deactivated catalyst comprising molecular sieve, particularly an OTO or olefin cracking process (OCP) catalyst, which does not require the cooling of the regeneration gas prior to scrubbing. Thus, OPEX savings are provided by dispensing with the cooling and reheating steps. This also provides CAPEX savings in terms of the pre-scrubbing cooling units which are no longer required. In addition, it is no longer necessary to dry the regeneration air after scrubbing because the present invention does not utilise an aqueous scrubbing system.

These benefits are achieved by treating the regeneration gas with a liquid absorbent stream comprising an ethylene glycol such as monoethylene glycol or an ethylene glycol oligomer to absorb at least a part of one or more contaminants present. In particular, the liquid absorbent stream can absorb at least a part of one or more of any water, any alkali metal ion and any alkaline earth metal ion present in the regeneration gas. This provides a treated regeneration gas which is depleted in one or more of any water, any alkali metal ion and any alkaline earth metal ion, without the need for cooling and reheating the regeneration gas. This treatment can be achieved without the heat losses associated with the cooling of the regeneration gas prior to aqueous scrubbing and the subsequent energy expended by reheating the scrubbed regeneration gas known from WO 2006/023189.

Reference herein to a deactivated catalyst comprising molecular sieve is to a catalyst comprising molecular sieve and comprising carbonaceous deposits. In oxygenate to olefin processes and olefin cracking processes, the catalyst comprising molecular sieve is typically deactivated due to the deposition of carbonaceous matter on the catalyst, in particular on the active sites of the molecular sieve in the catalyst. Therefore, preferably, the deactivated catalyst further comprises carbonaceous deposits.

In addition, treatment of the regeneration gas stream with the liquid absorbent stream may also lower the concentration of other contaminants in the treated regeneration gas compared to the untreated regeneration gas. Such other contaminants may be airborne pollutants, such as one or more selected from the group comprising solid particulates, such as soot, and oxides of sulphur, SOₓ.

Any ethylene glycol or ethylene glycol oligomer from the liquid absorbent stream which becomes entrained in the treated regeneration gas does not adversely affect the oxidative regeneration process and would be burned with the carbonaceous deposits. Furthermore, any residual ethylene glycol or ethylene glycol oligomer present on the regenerated catalyst returned to the reactor would not interfere with the conversion reaction, such as an OTO or OCP reaction, and indeed may be consumed as part of the feedstock of such processes.

In a first aspect, the present invention provides a process for the oxidative regeneration of a deactivated catalyst comprising molecular sieve wherein said deactivated catalyst is obtained from one or both of an oxygenate to olefin process and a olefin cracking process, to provide a regenerated catalyst comprising regenerated molecular sieve, said regeneration process comprising at least the steps of:

providing a regeneration gas stream comprising oxidant;

treating the regeneration gas stream with a liquid adsorbent stream comprising an ethylene glycol in a contaminant absorption zone to remove at least a part of one or more of any water, any alkali metal ion and any alkaline earth metal ion present in the regeneration gas stream to provide a treated regeneration gas stream comprising oxidant;

regenerating a deactivated catalyst comprising molecular sieve with the treated regeneration gas stream to provide a regenerated catalyst comprising molecular sieve.

In a one embodiment, the ethylene glycol may comprise one or more of monoethylene glycol and an ethylene glycol oligomer. The ethylene glycol oligomer may be formed of from two to ten monoethylene glycol monomers, more preferably two to four monoethylene glycol monomers. The ethylene glycol still more preferably comprises one or more of the group comprising mono ethylene glycol, diethylene glycol and triethylene glycol, most preferably monoethylene glycol.

In a further embodiment the alkali metal ion may be one or more selected from the group comprising Li⁺, Na⁺ and K⁺, particularly Na⁺. In a still further embodiment, the alkaline earth metal ion be one or more selected from the group comprising Mg²⁺, Ca²⁺, Sr²⁺ and Ba²⁺, more preferably Ca²⁺. Typically the metal ion comprises one or more of Li⁺, Na⁺, K⁺, Mg²⁺, Ca²⁺ and Sr²⁺, more typically Na⁺ and Ca²⁺.

In another embodiment, one or both of any alkali metal ion and alkaline earth metal ion may be present as a salt i.e. alkali metal salt or alkaline earth metal salt, such as NaCl and/or CaCl₂. The salt may be selected from one or more of the group comprising sodium chloride, lithium chloride, potassium chloride and calcium chloride.

The salt may be in the form of a solid salt and/or an aqueous solution of the salt. For instance, solid NaCl particles may be formed by the evaporation of water from saline, such as seawater, while the aqueous solution of NaCl may comprise droplets of seawater. The aqueous salt solution can be adsorbed by the liquid adsorbent stream. The solid salt particles can be removed from the regeneration gas into the liquid absorbent stream.

In yet another embodiment, the regeneration gas stream may further comprise one or more of water, alkali metal ion and alkaline earth metal ion, the treated regeneration gas stream may be depleted in one or more of water, alkali metal ion and alkaline earth metal ion and the step of treating the regeneration gas stream may further provide a spent liquid absorbent stream comprising an ethylene glycol, and one or more of water, alkali salt and alkaline earth salt. Typically, the regeneration gas stream may further comprise water and one or both of alkali metal ion and alkaline earth metal ion, the treated regeneration gas stream may be a water and one or both of alkali metal ion and alkaline earth metal ion depleted stream and the step of treating the regeneration gas stream may further provide a spent liquid absorbent stream comprising an ethylene glycol, water and one or both of alkali salt and alkaline earth salt.

In another embodiment, the deactivated catalyst from one or both of an oxygenate to olefin process and a olefin cracking process is provided by one or both of the steps of:

reacting an oxygenate feedstock comprising oxygenate in an oxygenate to olefin reaction zone in the presence of a catalyst comprising molecular sieve to produce a deactivated catalyst comprising molecular sieve and a reaction effluent stream comprising unreacted oxygenate, olefinic product and water; and

reacting an C4+ hydrocarbon feedstock comprising olefin in an olefin cracking process reaction zone in the presence of a catalyst comprising molecular sieve to produce the deactivated catalyst comprising molecular sieve and a reaction effluent stream comprising unreacted C4+ hydrocarbon and one or both of ethylene and propylene.

In yet another embodiment, the deactivated catalyst from one or both of an oxygenate to olefin process and a olefin cracking process is provided by the steps of:

(1) reacting an C4+ hydrocarbon feedstock comprising olefin in an olefin cracking process reaction zone in the presence of a catalyst comprising molecular sieve to produce the deactivated catalyst comprising molecular sieve and a reaction effluent stream comprising unreacted C4+ hydrocarbon and one or both of ethylene and propylene; and
(2) reacting an oxygenate feedstock comprising oxygenate in an oxygenate to olefin reaction zone in the presence of the deactivated catalyst of step (1) to produce deactivated catalyst comprising molecular sieve and a reaction effluent stream comprising unreacted oxygenate, olefinic product and water.

In a further embodiment, the process may further comprise the step of:

passing at least a portion of the spent liquid absorbent stream to the contaminant absorption zone as the liquid absorbent stream.

In a still further embodiment, the process may further comprise the steps of:

removing a portion of the spent liquid absorbent stream as a spent liquid absorbent bleed stream;

passing an ethylene glycol to the liquid absorbent stream as a liquid absorbent restoration stream. Typically, the ethylene glycol will have an identical composition of ethylene glycol(s) in the liquid absorbent stream.

In yet another embodiment, the process may further comprise the steps of:

drying the spent liquid absorbent stream to provide a regenerated liquid absorbent stream comprising an ethylene glycol and a rejection stream comprising water and one or both of alkali salt and alkaline earth salt;

passing the regenerated liquid absorbent stream to the contaminant absorption zone as the liquid absorbent stream.

In another embodiment, the process may further comprise, between the treating of the regeneration gas and the regeneration of the catalyst steps, the step of:

demisting the treated regeneration gas stream to remove at least a portion of any entrained ethylene glycol.

In yet another embodiment, the step of regenerating the deactivated catalyst may comprise oxidising the carbonaceous deposits with the treated regeneration gas stream.

In another embodiment, the step of regenerating the deactivated catalyst may be carried out at a temperature in the range of from 550 to 750° C., more preferably in the range of from 600 to 650° C.

In a further embodiment, one or both of the temperature and mass flow of the treated regeneration gas stream may be controlled to control the rate of the oxidative regeneration of the deactivated catalyst in the regeneration step. The oxidation of carbonaceous material such as coke is exothermic such that the rate of the reaction can be controlled by manipulating the reaction temperature by controlling the temperature of the treated regeneration gas.

In a further embodiment, the process may further comprise, prior to passing the regeneration gas stream to the contaminant absorption zone, the step of:

heating the regeneration gas stream to provide a heated regeneration gas stream; such that the treated regeneration gas stream is a heated stream.

In a still further embodiment, the process may further comprise, between the step of passing the regeneration gas stream to the contaminant absorption zone and the step of regenerating the deactivated catalyst, the step of:

heating the treated regeneration gas stream.

The step of heating the treated regeneration gas stream provides a treated regeneration gas stream as a heated stream.

In another embodiment, the oxidant in the regeneration gas stream may comprise oxygen. In a further embodiment, the regeneration gas stream may comprise air. Typically the regeneration gas stream is an air stream.

In yet another embodiment, the molecular sieve may be selected from the group comprising silicoaluminophosphate and aluminosilicate. The molecular sieve may be an aluminosilicate having at least a 10-membered ring zeolite structure. The molecular sieve may comprise one or more of the group comprising a TON-type aluminosilicate, such as ZSM-22, a MTT-type aluminosilicate, such as ZSM-23, and a MFI-type aluminosilicate, such as ZSM-5. Typically the molecular sieve is at least partially in hydrogen form.

In a second aspect, the present invention provides a process for the preparation of olefinic product, the process comprising at least the steps of:
(a) reacting an oxygenate feedstock comprising oxygenate in an oxygenate to olefin reaction zone in the presence of a catalyst comprising molecular sieve to produce a deactivated catalyst comprising molecular sieve and a reaction effluent stream comprising unreacted oxygenate, olefinic product and water;
(b) regenerating the deactivated catalyst according to a process of the first aspect and its embodiments discussed above to provide a regenerated catalyst.

The olefinic product typically comprises one or more of ethylene, propylene, butylene(s) and pentylene(s). Preferably the olefinic product comprises ethylene and optionally one or more of propylene, butylene(s) and pentylene(s).

In a third aspect, the present invention provides a process for the preparation of olefinic product comprising one or both of ethylene and propylene, the process comprising at least the steps of:
(a) reacting an C4+ hydrocarbon feedstock comprising olefin in an olefin cracking process reaction zone in the presence of a catalyst comprising molecular sieve to produce a deactivated catalyst comprising molecular sieve and a reaction effluent stream comprising unreacted C4+ hydrocarbon and one or both of ethylene and propylene;
(b) regenerating the deactivated catalyst according to a process of the first aspect and its embodiments discussed above to provide a regenerated catalyst.

In a fourth aspect, the present invention provides, a process for the preparation of olefinic product, the process comprising at least the steps of:
(a) (1) reacting an C4+ hydrocarbon feedstock comprising olefins in an olefin cracking process reaction zone in the presence of a catalyst comprising molecular sieve to produce a deactivated catalyst comprising molecular sieve and a reaction effluent stream comprising unreacted C4+ hydrocarbon and one or both of ethylene and propylene;
   (2) reacting an oxygenate feedstock comprising oxygenate in an oxygenate to olefin reaction zone in the presence of the deactivated catalyst of step (1) to produce deactivated catalyst comprising molecular sieve and a reaction effluent stream comprising unreacted oxygenate, olefinic product and water
(b) regenerating the deactivated catalyst according to a process of the first aspect and its embodiments discussed above to provide a regenerated catalyst.

In one embodiment of the third or fourth aspects, the molecular sieve is aluminosilicate molecular sieve, and more particularly zeolite molecular sieve.

In one embodiment of the second, third or fourth aspects, the process may further comprise the steps of:
(c) repeating step (a) at least once with the regenerated catalyst;
(d) repeating step (b) at least once with the deactivated catalyst.

In a fifth aspect, the present invention provides an apparatus for the oxidative regeneration of a deactivated catalyst comprising molecular sieve to provide a regenerated catalyst comprising regenerated molecular sieve, said apparatus comprising at least:

a contaminant absorption zone comprising a liquid absorbent, said liquid absorbent comprising an ethylene glycol, said contaminant absorption zone having a first inlet for a regeneration gas stream comprising oxidant and a first outlet for a treated regeneration gas stream comprising oxidant, said first outlet in fluid communication with the first inlet of a regeneration zone, said contaminant absorption zone further comprising a second inlet for a liquid absorption stream and a second outlet for a spent liquid absorption stream;

a regeneration zone comprising a deactivated catalyst comprising molecular sieve, said deactivated catalyst from one or both of an oxygenate to olefin process and a olefin cracking process, said regeneration zone having a first inlet for the treated regeneration gas stream and a first outlet for a regeneration effluent stream.

In one embodiment of the fourth aspect, the regeneration zone may be an OTO or OCP reaction zone, particularly an OTO or OCP reactor.

In another embodiment of the fifth aspect, the regeneration zone may further comprise a second outlet for a fluidised regenerated catalyst stream, said second outlet in fluid communication with a first inlet of an reaction zone, and a second inlet for a fluidised deactivated catalyst stream in fluid communication with a first outlet of the reaction zone, and

a OTO or OCP reaction zone having a first inlet for the fluidised regenerated catalyst stream, a first outlet for the deactivated catalyst fluid stream, a second outlet for a reaction effluent stream and a second inlet for a feedstock stream.

In a further embodiment of the fifth aspect, the reaction zone may be a reactor, such as a fluidised bed or riser reactor.

### DETAILED DESCRIPTION OF THE INVENTION

Embodiments of the present invention will now be described by way of example only and with reference to the accompanying non-limited drawing in which:

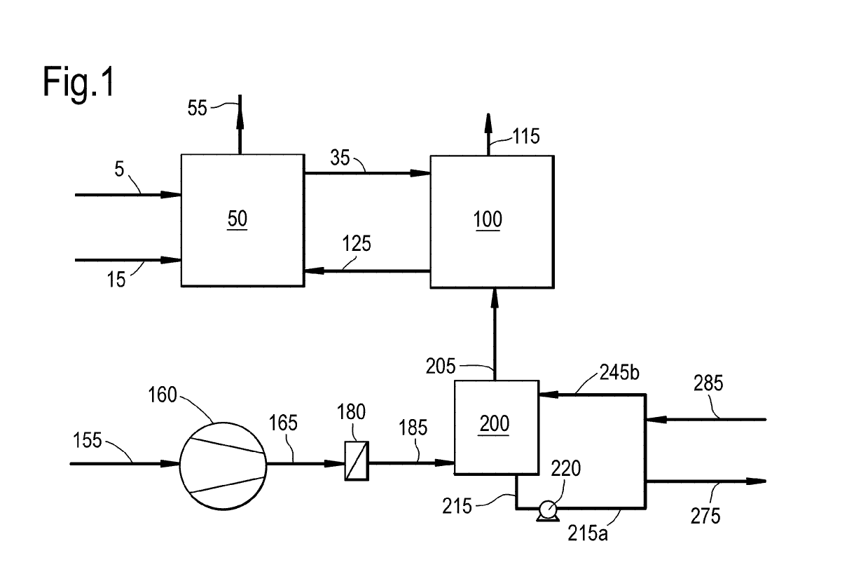

FIG. 1 is a diagrammatic scheme of one embodiment of a process for the oxidative regeneration of a deactivated OTO catalyst described herein.

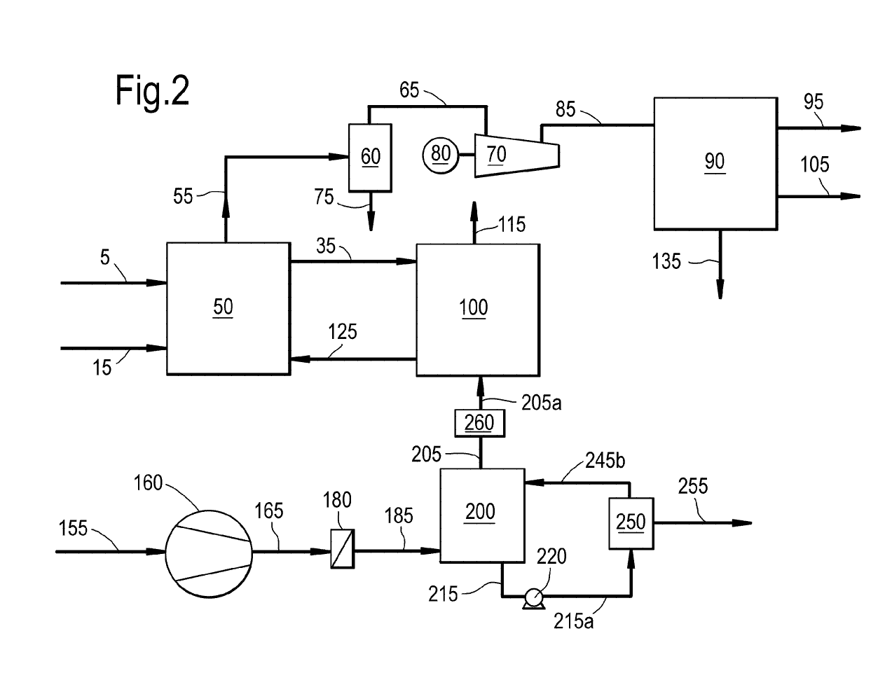

FIG. 2 is a diagrammatic scheme of another embodiment of a process for the oxidative regeneration of a deactivated OTO catalyst described herein.

FIG. 1 shows a diagrammatic scheme of a first embodiment of a process for the oxidative regeneration of a deactivated OTO catalyst comprising molecular sieve.

The deactivated catalyst regenerated in the process and apparatus described herein can be produced during the catalytic conversion of an oxygenate feedstock to olefinic products in an oxygenate-to-olefins process or during the catalytic cracking of a C4+ hydrocarbon feedstock comprising olefin in an olefin cracking process.

For instance, FIG. 1 shows an oxygenate feedstock stream 5 can be contacted in an OTO reaction zone 50, such as an OTO reactor, with a catalyst for oxygenate conversion under oxygenate conversion conditions, to obtain a reaction effluent comprising olefins, particularly lower olefins. Reference herein to an oxygenate feedstock is to an oxygenate-comprising feedstock. In the OTO reaction zone 50, at least part of the feedstock is converted into a product containing one or more olefins, preferably including lower olefins, in particular ethylene and typically propylene. The reaction effluent can be removed from OTO reaction zone 50 as reaction effluent stream 55. Reaction effluent stream 55 may comprise unreacted oxygenate, olefinic product and water and can be processed in a variety of ways known in the art. The embodiment of FIG. 2 discussed below shows one preferred line-up for the processing of the reaction effluent.

The oxygenate used in the process is preferably an oxygenate which comprises at least one oxygen-bonded alkyl group. The alkyl group preferably is a C1-C5 alkyl group, more preferably C1-C4 alkyl group, i.e. comprises 1 to 5, or 1 to 4 carbon atoms respectively; more preferably the alkyl group comprises 1 or 2 carbon atoms and most preferably one carbon atom. Examples of oxygenates that can be used in the oxygenate feedstock include alcohols and ethers. Examples of preferred oxygenates include alcohols, such as methanol, ethanol, propanol; and dialkyl ethers, such as dimethylether, diethylether, methylethylether. Preferably, the oxygenate is methanol or dimethylether, or a mixture thereof.

Preferably the oxygenate feedstock comprises at least 50 wt % of oxygenate, in particular methanol and/or dimethylether, based on total hydrocarbons, more preferably at least 70 wt %.

The oxygenate feedstock can comprise an amount of diluent, such as water or steam. In one embodiment, the molar ratio of oxygenate to diluent is between 10:1 and 1:10, preferably between 4:1 and 1:2, in particular when the oxygenate is methanol and the diluent is water (typically steam).

Preferably, in addition to the oxygenate, an olefinic co-feed is provided along with and/or as part of the oxygenate feedstock. FIG. 1 shows the co-feed being supplied to OTO reaction zone 50 as an olefinic co-feed stream 15. Reference herein to an olefinic co-feed is to an olefin-comprising co-feed. The olefinic co-feed preferably comprises C4 and higher olefins, more preferably C4 and C5 olefins. Preferably, the olefinic co-feed comprises at least 25 wt %, more preferably at least 50 wt %, of C4 olefins, and at least a total of 70 wt % of C4 hydrocarbon species.

Preferably, at least 70 wt % of the olefinic co-feed, during normal operation, is formed by a recycle stream of a C4+ hydrocarbon fraction from the OTO reaction effluent. Preferably at least 90 wt % of olefinic co-feed, based on the whole olefinic co-feed, is formed by such recycle stream. In order to maximize production of ethylene and propylene, it is desirable to maximize the recycle of C4 olefins in the effluent of the OTO process. This can be done by recycling at least part of the C4+ hydrocarbon fraction, preferably C4-C5 hydrocarbon fraction, more preferably C4 hydrocarbon fraction, in the OTO effluent. However, a certain part thereof, such as between 1 and 5 wt %, can be withdrawn as purge, since otherwise saturated hydrocarbons, in particular C4s (butane) would build up in the process, which are substantially not converted under the OTO reaction conditions.

The preferred molar ratio of oxygenate in the oxygenate feedstock to olefin in the olefinic co-feed provided to the OTO conversion zone depends on the specific oxygenate used and the number of reactive oxygen-bonded alkyl groups therein. Preferably the molar ratio of oxygenate to olefin in the total feed lies in the range of 20:1 to 1:10, more preferably in the range of 18:1 to 1:5, still more preferably in the range of 15:1 to 1:3, even still more preferably in the range of 12:1 to 1:3.

A variety of OTO processes are known for converting oxygenates, such as for instance methanol or dimethylether to an olefin-containing product, as already referred to above. One such process is described in WO-A 2006/020083. Processes integrating the production of oxygenates from synthesis gas and their conversion to light olefins are described in US20070203380A1 and US20070155999A1.

Catalysts suitable for converting the oxygenate feedstock comprise molecular sieve. Such molecular sieve-comprising catalysts typically also include binder materials, matrix material and optionally fillers. Suitable matrix materials include clays, such as kaolin. Suitable binder materials include silica, alumina, silica-alumina, titania and zirconia, wherein silica is preferred due to its low acidity.

Molecular sieves preferably have a molecular framework of one, preferably two or more corner-sharing tetrahedral units, more preferably, two or more [SiO₄], [AlO₄] and/or [PO₄] tetrahedral units. These silicon, aluminum and/or phosphorus based molecular sieves and metal containing silicon, aluminum and/or phosphorus based molecular sieves have been described in detail in numerous publications including for example, U.S. Pat. No. 4,567,029. In a preferred embodiment, the molecular sieves have 8-, 10- or 12-ring structures and an average pore size in the range of from about 3 Å to 15 Å.

Suitable molecular sieves are silicoaluminophosphates (SAPO), such as SAPO-17, -18, -34, -35, -44, but also SAPO-5, -8, -11, -20, -31, -36, -37, -40, -41, -42, -47 and -56; aluminophosphates (AlPO) and metal substituted (silico)aluminophosphates (MeAlPO), wherein the Me in MeAlPO refers to a substituted metal atom, including metal selected from one of Group IA, IIA, IB, IIIB, IVB, VB, VIB, VIIB, VIIIB and Lanthanides of the Periodic Table of Elements. Preferably Me is selected from one of the group consisting of Co, Cr, Cu, Fe, Ga, Ge, Mg, Mn, Ni, Sn, Ti, Zn and Zr.

Alternatively, the conversion of the oxygenate feedstock may be accomplished by the use of an aluminosilicate-comprising catalyst, in particular a zeolite-comprising catalyst. Suitable catalysts include those containing a zeolite of the ZSM group, in particular of the MFI type, such as ZSM-5, the MTT type, such as ZSM-23, the TON type, such as ZSM-22, the MEL type, such as ZSM-11, and the FER type. Other suitable zeolites are for example zeolites of the STF-type, such as SSZ-35, the SFF type, such as SSZ-44 and the EU-2 type, such as ZSM-48.

Aluminosilicate-comprising catalyst, and in particular zeolite-comprising catalyst are preferred when an olefinic co-feed is fed to the oxygenate conversion zone together with oxygenate, for increased production of ethylene and propylene.

Preferred catalysts comprise a more-dimensional zeolite, in particular of the MFI type, more in particular ZSM-5, or of the MEL type, such as zeolite ZSM-11. Such zeolites are particularly suitable for converting olefins, including iso-olefins, to ethylene and/or propylene. The zeolite having more-dimensional channels has intersecting channels in at least two directions. So, for example, the channel structure is formed of substantially parallel channels in a first direction, and substantially parallel channels in a second direction, wherein channels in the first and second directions intersect. Intersections with a further channel type are also possible. Preferably, the channels in at least one of the directions are 10-membered ring channels. A preferred MFI-type zeolite has a silica-to-alumina ratio, SAR, of at least 60, preferably at least 80. Preferred catalysts may include catalysts comprising one or more zeolites having one-dimensional 10-membered ring channels, i.e. one-dimensional 10-membered ring channels, which are not intersected by other channels. Preferred examples are zeolites of the MTT and/or TON type.

In a particularly embodiment the catalyst comprises in addition to one or more one-dimensional zeolites having 10-membered ring channels, such as of the MTT and/or TON type, a more-dimensional zeolite, in particular of the MFI type, more in particular ZSM-5, or of the MEL type, such as zeolite ZSM-11.

The catalyst may further comprise phosphorus as such or in a compound, i.e. phosphorus other than any phosphorus included in the framework of the molecular sieve. It is preferred that a MEL or MFI-type zeolite comprising catalyst additionally comprises phosphorus. The phosphorus may be introduced by pre-treating the MEL or MFI-type zeolites prior to formulating the catalyst and/or by post-treating the formulated catalyst comprising the MEL or MFI-type zeolites. Preferably, the catalyst comprising MEL or MFI-type zeolites comprises phosphorus as such or in a compound in an elemental amount of from 0.05-10 wt % based on the weight of the formulated catalyst. A particularly preferred catalyst comprises phosphorus-treated MEL or MFI-type zeolite having SAR of in the range of from 60 to 150, more preferably of from 80 to 100. An even more particularly preferred catalyst comprises phosphorus-treated ZSM-5 having SAR of in the range of from 60 to 150, more preferably of from 80 to 100.

It is preferred that the molecular sieves in the hydrogen form are used in the oxygenate conversion catalyst, e.g., HZSM-22, HZSM-23, and HZSM-48, HZSM-5. Preferably at least 50% w/w, more preferably at least 90% w/w, still more preferably at least 95% w/w and most preferably 100% of the total amount of molecular sieve used is in the hydrogen form. It is well known in the art how to produce such molecular sieves in the hydrogen form.

The reaction conditions of the oxygenate conversion in step (a) of the second aspect and step (a)(2) of the fourth aspect, include a reaction temperature of 350 to 1000° C., preferably from 350 to 750° C., more preferably 450 to 700° C., even more preferably 500 to 650° C.; and a pressure from 0.1 kPa (1 mbar) to 5 MPa (50 bar), preferably from 100 kPa (1 bar) to 1.5 MPa (15 bar).

Preferably, the oxygenate feedstock is preheated to a temperature in the range of from 200 to 550° C., more preferably 250 to 500° C. prior to contacting with the molecular sieve-comprising catalyst.

The catalyst particles used in the process can have any shape known to the skilled person to be suitable for this purpose, and can be present in the form of spray dried catalyst particles, spheres, tablets, rings, extrudates, etc. Extruded catalysts can be applied in various shapes, such as, cylinders and trilobes. Spray-dried particles allowing use in a fluidized bed or riser reactor system are preferred. Spherical particles are normally obtained by spray drying. Preferably the average particle size is in the range of 1-200 μm, preferably 50-100 μm, still more preferably approximately 70 μm.

Although the C4+ hydrocarbon fraction in the reaction effluent may be recycled as an olefinic co-feed (e.g. stream 15), in an alternative embodiment not shown in FIG. 1, at least part of the olefins in the C4+ hydrocarbon fraction are converted to ethylene and/or propylene by contacting the C4+ hydrocarbon fraction in a separate unit with a molecular sieve-comprising catalyst, particularly a zeolite-comprising catalyst. This is particularly preferred where molecular sieve-comprising catalyst in the OTO process comprises a least one SAPO, ALPO, or MeAlPO type molecular sieve, preferably SAPO-34. These catalysts are less suitable for converting olefins. Preferably, the C4+ hydrocarbon fraction is contacted with the zeolite-comprising catalyst at a reaction temperature of 350 to 1000° C., preferably from 375 to 750° C., more preferably 450 to 700° C., even more preferably 500 to 650° C.; and a pressure from 0.1 kPa (1 mbar) to 5 MPa (50 bar), preferably from 100 kPa (1 bar) to 1.5 MPa (15 bar). Optionally, the stream comprising C4+ olefins also contains a diluent. Examples of suitable diluents include, but are not limited to, liquid water or steam, nitrogen, argon, paraffins and methane. Under these conditions, at least part of the olefins in the C4+ hydrocarbon fraction are converted to further ethylene and/or propylene. The further ethylene and/or propylene may be combined with the further ethylene and/or propylene obtained directly from the OTO reaction zone. Such a separate process step directed at converting C4+ olefins to ethylene and propylene is also referred to as an olefin cracking process (OCP).

Catalysts comprising molecular sieve, particularly aluminosilicate-comprising catalysts, and more particularly zeolite-comprising catalysts, have the further advantage that in addition to the conversion of methanol or ethanol, these catalysts also induce the conversion of olefins to ethylene and/or propylene. Therefore, aluminosilicate-comprising catalysts, and in particular zeolite-comprising catalysts, are particularly suitable for use as the catalyst in an OCP. The preferences provided herein above for the oxygenate to olefins catalyst apply mutatis mutandis for the OCP catalyst with the primary exception that the OCP catalyst always comprises at least one zeolite.

Particular preferred catalysts for the OCP reaction, i.e. converting part of the olefinic product, and preferably part of the C4+ hydrocarbon fraction of the olefinic product including olefins, are catalysts comprising at least one zeolite selected from MFI, MEL, TON and MTT type zeolites, more preferably at least one of ZSM-5, ZSM-11, ZSM-22 and ZSM-23 zeolites.

The catalyst may further comprise phosphorus as such or in a compound, i.e. phosphorus other than any phosphorus included in the framework of the molecular sieve. It is preferred that a MEL or MFI-type zeolite comprising catalyst additionally comprises phosphorus. The phosphorus may be introduced by pre-treating the MEL or MFI-type zeolites prior to formulating the catalyst and/or by post-treating the formulated catalyst comprising the MEL or MFI-type zeolites. Preferably, the catalyst comprising MEL or MFI-type zeolites comprises phosphorus as such or in a compound in an elemental amount of from 0.05-10 wt % based on the weight of the formulated catalyst. A particularly preferred catalyst comprises phosphorus-treated MEL or MFI-type zeolite having SAR of in the range of from 60 to 150, more preferably of from 80 to 100. An even more particularly preferred catalyst comprises phosphorus-treated ZSM-5 having SAR of in the range of from 60 to 150, more preferably of from 80 to 100.

Preferably, the oxygenate to olefins catalyst and the olefin cracking catslyst are the same zeolite comprising catalyst.

Both the OTO process and the OCP may be operated in a fluidized bed or moving bed, e.g. a fast fluidized bed or a riser reactor system, and also in a fixed bed reactor or a tubular reactor. A fluidized bed or moving bed, e.g. a fast fluidized bed or a riser reactor system are preferred.

In a further embodiment not shown in FIG. 1, catalyst comprising molecular sieve may be first used in an OCP reaction zone for the conversion of the C4+ olefins of the C4+ hydrocarbon fraction, and subsequently transferred to the OTO reaction zone for conversion of the oxygenate feedstock stream 5 and olefinic co-feed stream 15.

As discussed above, the catalyst can deactivate in the course of the OTO process and OCP to produce deactivated catalyst comprising molecular sieve. The deactivation occurs primarily due to deposition of carbonaceous deposits, such as coke, on the catalyst by side reactions, to produce deactivated molecular sieve comprising carbonaceous deposits. The carbonaceous deposits can block the access of the oxygenate feedstock to the active sites of the molecular sieve.

The deactivated catalyst can be regenerated to remove a portion of the carbonaceous deposit such as coke. It is not necessary, and indeed may be undesirable, to remove all the carbonaceous deposit from the catalyst as it is believed that a small amount of residual carbonaceous deposit such as coke may enhance the catalyst performance. Additionally, it is believed that complete removal of the carbonaceous deposit may also lead to degradation of the molecular sieve.

The same catalyst may be used for both the OTO process and OCP. In such a situation, the catalyst comprising molecular sieve, particularly comprising aluminosilicate molecular sieve and more particularly comprising zeolite, may be first used in the OCP. The deactivated catalyst from the OCP may then be used, typically without oxidative regeneration, in the OTO process. The deactivated catalyst from the OTO process may then be regenerated as described herein, and the regenerated catalyst then used again in the OCP.

This line-up may be beneficial because it provides good heat integration between the OCP, OTO and oxidative regeneration processes. The OCP is endothermic and at least a portion of the heat of reaction can be provided by passing catalyst from the regeneration zone to the OCP reaction zone, because the regeneration reaction which oxidizes the carbonaceous deposits from the deactivated catalyst is exothermic.

In the process described herein, the deactivated catalyst comprising molecular sieve can be regenerated with a treated regeneration gas stream 205. In the embodiment shown in FIG. 1, deactivated catalyst is transferred from the reaction zone 50 to a regeneration zone 100, for instance as a fluidised deactivated catalyst stream 35.

The regeneration zone 100 may be a regenerator such as a fluidized bed or moving bed, e.g. a fast fluidized bed or a riser reactor system. Oxidative regeneration may also be carried out in the reaction zone, particularly the reactor itself, such as in a fixed bed reactor or a tubular reactor. In an embodiment not shown in FIG. 1, the oxidative regeneration may be carried out in the reaction zone 50, although this is not preferred.

Oxidative regeneration of the deactivated catalyst comprising molecular sieve is carried out with a treated regeneration gas stream 205. The treated regeneration gas stream 205 can comprise an oxidant, such as oxygen. Typically, the stream may comprise treated air. The stream has been treated in order to reduce the concentration of one or more of any water, any alkali metal ions and any alkaline earth metal ions present.

Preferably the stream has been treated to reduce the concentration of any water and one or both of any alkali metal ions and any alkaline earth metal ions present because the contaminant ions are commonly present in aqueous solution, for instance in the form of water droplets.

Water present in the regeneration gas may hydrothermally damage the molecular sieve at the regeneration temperature. In addition, alkali metal ions and alkaline earth metal ions can ion exchange with a molecular sieve during oxidative regeneration, particularly a molecular sieve in hydrogen form, such as typical catalysts for OTO process and OCP, resulting in the deactivation of catalytic activity by the removal of active sites, particularly acidic sites. The loss of active sited on the molecular sieve leads to a decrease in the catalytic activity of the regenerated catalyst comprising the regenerated molecular sieve i.e. oxidative regeneration cannot reverse the deactivation of active sites in hydrogen form by ion exchange. For this reason, catalyst comprising ion exchanged molecular sieve is referred to herein as spent catalyst.

Thus, the treated regeneration gas stream 205 should be a water, an alkali metal ion and/or alkaline earth metal ion depleted treated regeneration gas stream. In the present context, the term “depleted” means that the concentration of the depleted component is less than the corresponding untreated stream. Thus, a water, alkali metal ion and alkaline earth metal ion depleted treated regeneration gas stream would have reduced concentrations of water, alkali metal ion and alkaline earth metal ion compared to the stream prior to treatment (i.e. regeneration gas stream 185).

In a preferred embodiment, the treated regeneration gas stream 205 comprises less than 500 wt ppb total alkali metal ion and alkaline earth metal ion, more preferably, the less than 300 wt ppb total, still more preferably less than 100 wt ppb total based on the treated regeneration gas stream 205. In another preferred embodiment, the treated regeneration gas stream 205 comprises less than 250 wt ppb sodium ion, more preferably less than 150 wt ppb, still more preferably less than 50 wt ppb based on the treated regeneration gas stream 205. In yet another preferred embodiment, the treated generated gas stream 205 comprises less than 4 mol % water, more preferably less than 3 mol %, still more preferably less than 2 mol % based on the treated regeneration gas stream 205.

The regeneration process can heat the molecular sieve in an oxidising environment supplied by the treated regeneration gas stream. The regeneration step can oxidise the carbonaceous deposits on the molecular sieve to produce gaseous oxides of carbon, which can leave the regeneration zone 100 as regeneration effluent stream 115. The regeneration effluent stream 115 may comprise one or more oxidation products of the carbonaceous material, such as carbon monoxide and carbon dioxide, as well as any unreacted components of the treated regeneration gas stream, such as any unreacted oxidant, and any inert components, such as nitrogen and/or carbon dioxide already present in the treated regeneration gas stream 205 e.g. if air is used.

The oxidative regeneration step can be carried out at a regeneration temperature in the range of from 550 to 750° C., more preferably in the range of from 600 to 650° C. The oxidative regeneration step can be initiated by heating the regeneration zone 100 to regeneration temperature in the presence of the treated regeneration gas stream 205. This can be achieved, for instance, by pre-heating the treated regeneration gas stream 205, such that it is provided as a heated stream, such as a temperature at or above 550° C., to initiate oxidation of the carbonaceous material on the molecular sieve. Once the oxidative regeneration reaction is underway, the regeneration gas stream 205 may be heated to a temperature of about 250 to 300° C., more typically approximately 250° C. to maintain the reaction. Alternatively or additionally, the regeneration zone 100 itself may be heated, for instance by heating elements.

The oxidation of carbonaceous material such as coke which occurs during regeneration is exothermic such that controlling one or both of the temperature and the mass flow of the treated regeneration gas stream 205 can be used to control the regeneration temperature, and therefore rate of the regeneration reaction.

Typically the mass flow of the treated regeneration gas stream 205 is adjusted to achieve a desired reduction in the level of carbonaceous material, such as coke, on the molecular sieve. This is known as the “delta coke level”. The delta coke level is proportional to the heat that is generated by the exothermic regeneration reaction. The oxidative regeneration step may be carried out in a “full burn” mode in which substantially all the carbonaceous deposits are removed by conversion into carbon dioxide. Alternatively, the oxidative regeneration step may be carried out in “half burn” mode in which more carbonaceous deposit remains on the molecular sieve after regeneration, and mostly carbon monoxide is produced. These two oxidative regeneration modes result in different changes in enthalpy and therefore different changes in temperature. For a given oxidative regeneration mode, the change in temperature can therefore be used as a measure of the extent of coke removal.

It will be apparent that the oxidative regeneration reaction can be terminated by lowering the temperature of the treated regeneration gas stream 205, for instance to a temperature below 250° C., more preferably below 200° C., still more preferably below 150° C., such that the temperature of the molecular sieve in the regeneration zone 100 is reduced below that required to support combustion of the carbonaceous material.

Once the molecular sieve has been heated for a sufficient time to remove at least a portion of the carbonaceous material, the regeneration reaction can be terminated and the regenerated catalyst comprising molecular sieve can be returned to the reaction zone 50, for instance as fluidised regenerated catalyst stream 125. In this way, batch wise or continuous oxidative regeneration processes may be started-up and shut down.

However, in a continuous oxidative regeneration process, such as that shown in the embodiment of FIG. 1, the regeneration zone may be continuously supplied with deactivated catalyst from the reaction zone 50 via fluidised deactivated catalyst stream 35. Similarly, fluidised regenerated catalyst stream 125 may be continuously returned to the reaction zone 50. The regeneration zone 100 can be operated at a pre-selected level of delta coke with a balanced mass flow of incoming and outgoing catalyst via fluidised catalyst streams 35 and 125. The regeneration temperature can be controlled by adjusting the mass flow of regeneration gas, particularly treated regeneration gas stream 205 and/or heated treated regeneration gas stream 205a provided as discussed below for the embodiment of FIG. 2.

The treated regeneration gas stream 205 is formed by the treatment of a regeneration gas stream 185. The regeneration gas stream 185 comprises an oxidant. The oxidant may be one or more of the group selected from oxygen, ozone, sulphur trioxide, and NOₓ (such as N₂O, NO, NO₂ and/or N₂O₅). Oxygen is preferred, such that the regeneration gas stream 185 may comprise air. The air may be diluted with a diluent such as nitrogen or carbon dioxide, if required.

The regeneration gas stream 185 may comprise contaminants, some of which, like water, alkali metal ions and alkaline earth metal ions, may be damaging to the molecular sieve to be regenerated. Thus, in this context, water, alkali metal ions and alkaline earth metal ions can all be viewed as contaminants which can be detrimental to the activity of the catalyst i.e. catalyst poisons. These contaminants may occur in the regeneration gas stream 185 if, for instance, the regeneration zone 100 is located in a coastal environment, such that regeneration gas comprising air may be contaminated with salt (NaCl) or saline water.

The concentration of any such contaminants in the regeneration gas can be reduced by treating regeneration gas stream 185 with a liquid absorbent stream 245 comprising an ethylene glycol in a contaminant absorption zone 200. The ethylene glycol may be one or more of monoethylene glycol and ethylene glycol oligomers. The ethylene glycol oligomer is preferably selected from one or more of the group comprising diethylene glycol, triethylene glycol and tetraethylene glycol.

The contaminant absorption zone 200 can facilitate the intimate contact of the liquid absorbent stream 245 with the regeneration gas stream 185, thereby allowing the absorption of at least a portion of any contaminants present such as one or more of alkali metal ion, alkaline earth metal ion and water to provide treated regeneration gas stream 205. The contaminant absorption zone 200 may be a gas-liquid contactor, such as a column comprising packing and/or a plurality of trays.

The monoethylene glycol and/or ethylene glycol oligomers also absorb alkali metal ions and alkaline earth metal ions in particulate form, for instance when they are present in the regeneration gas as entrained solid salts, or they can absorb aqueous solutions of such ions, such as aerosols of such solutions dispersed in the regeneration gas. Monoethylene glycol is a preferred component of the liquid absorbent because it is miscible with water in all proportions, has a boiling point of 193° C., and a low vapour pressure.

Although the presence of amounts of liquid absorbent in the treated regeneration gas stream 205 should not affect the oxidative regeneration process, the treated regeneration gas stream can be passed through a demister (not shown) to remove any entrained liquid absorbent.

It will be apparent that treating the regeneration gas stream 185 with a liquid absorbent steam 245 can also remove other contaminants, such as particulate material like dust and/or soot entrained in the regeneration gas stream.

The removal of contaminants from the regeneration gas stream 185 into the liquid absorbent produces a spent liquid absorbent stream 215, typically at or near the bottom of the contaminant absorption zone 200. Such a spent liquid absorbent stream 215 may comprise, in addition to an ethylene glycol, at least a portion of any contaminants present in the regeneration gas stream 185, such as one or more of any water, alkali metal ions and alkaline earth metal ions and optionally other contaminants, such as particulate material. The spent liquid absorbent stream 215 may be returned directly to the contaminant absorption zone 200 to treat further regeneration gas, for instance by liquid absorbent pump 220, which produces pumped spent liquid absorbent stream 215a.

However, it will be apparent that absorbed contaminants will accumulate in the recycled spent liquid absorbent. In order to control the level of contaminants in the spent liquid absorbent returned to the contaminant absorption zone 200, a portion of pumped spent liquid absorbent stream 215a may be drawn off as spent liquid absorbent bleed stream 275, thereby providing continuing spent liquid absorbent stream 215b as the remaining portion. The continuing spent liquid absorbent stream 215b can be supplemented with fresh liquid absorbent via liquid absorbent restoration stream 285, to provide liquid absorbent stream 245 which is supplied to the contaminant absorption zone 200. In this embodiment, the liquid absorbent stream 245 therefore comprises spent liquid absorbent and fresh liquid absorbent.

As used herein, the term “fresh liquid absorbent” means liquid absorbent which has not previously been used to treat the regeneration gas stream or the product of the purification of spent liquid absorbent which has been treated to reduce the concentration of contaminants therein. Means to purify the spent liquid absorbent, and particularly spent liquid absorbent bleed stream 275 to provide liquid absorbent restoration stream 285 is discussed in the embodiment of FIG. 2 below, in relation to drying apparatus 250.

It is preferred that the liquid absorbent restoration stream 285 is substantially free of nitrogen- and/or sulphur-containing impurities. In the present context, the term “substantially free” means that such impurities are present in trace amounts e.g. less than 100 ppm by weight of the total liquid absorbent stream. This is advantageous because if any liquid absorbent is carried into regenerator 100 from contaminant absorption zone 200, it could be oxidised during the regeneration step. Nitrogen- and sulphur-containing impurities can be oxidised to oxides of nitrogen (NOx) and oxides of sulphur (SOx) respectively, which are gaseous pollutants which would exit the regeneration zone 100 in the regeneration effluent stream 115. A regeneration effluent stream 115 comprising one or both of oxides of nitrogen and oxides of sulphur may require further treatment to remove such pollutants. Consequently, it is beneficial that the liquid absorbent restoration stream 285 is substantially free of such impurities.

Similarly, it is also preferred that the regeneration gas does not comprise oxides of nitrogen or oxides of sulphur, in order to avoid their contamination of the regeneration effluent stream 115. For this reason sulphur trioxide and NOₓ are not preferred oxidants for the regeneration gas.

Prior to treatment in the contaminant absorption zone 200, the regeneration gas stream 185 may have been filtered to remove solid particulates. In the embodiment of FIG. 1, a pressurised regeneration gas stream 165 is passed through particulate removal means 180, in which solid particulates are removed to provide the regeneration gas stream 185. Typically, the particulate removal means 180 can remove particles with a particle size of greater than 30 micrometers, more typically greater than 10 micrometers. The particulate removal means may be one or more selected from the group comprising screens or meshes, inertial separators, viscous impingement filters and barrier filters.

Such a filtration step can remove solid particulates such as dust and solids comprising salt (NaCl) from the regeneration gas before it is treated with the liquid absorbent stream 245 in the contaminant absorption zone 200, thereby preventing them from building up in the spent liquid absorbent stream 215. This, in this embodiment the regeneration gas stream 185 is a solid particulate depleted stream.

The pressurised regeneration gas stream 165 may be provided by a blower 160, or other pressurising means such as a compressor, which is supplied with a regeneration gas feed stream 155 at its suction. The pressurising means should provide sufficient pressure to pass the regeneration gas to the regeneration zone 100. Typically the regeneration zone will operate at a pressure of approximately 1 bar.

If the pressurising means 160 provides the pressurised regeneration gas stream 165 at a temperature above that desirable for downstream equipment or steps, it may be cooled by known systems in the art, such as an air or water cooler. Alternatively, the regeneration gas stream may be cooled in the contaminant absorption zone 200 by providing the liquid absorbent stream 245 as a cooled steam, for instance by passing it through a liquid absorbent heat exchanger (not shown), such as an air or water cooler.

The regeneration gas feed stream 155, may comprise air or another suitable oxidant-comprising stream drawn into blower 160.

FIG. 2 provides an alternative embodiment of the process and apparatus described herein. Identical reference numerals to the embodiment of FIG. 1 correspond to identical streams or equipment.

The regeneration gas feed stream 155 can blown to particulate removal means 180 as pressurised regeneration gas stream 165, to provide regeneration gas stream 185 in a similar manner to the embodiment of FIG. 1. In the contaminant absorption zone 200, the regeneration gas stream 185 is contacted with a liquid absorbent stream 245b comprising an ethylene glycol to provide treated regeneration gas stream 205 and spent liquid absorbent stream 215. The spent liquid absorbent stream can be passed to the suction of a liquid absorbent pump 220, to provide a pumped spent liquid absorption stream 215a at its discharge. It will be apparent that the liquid absorbent pump 220 can be placed elsewhere in the liquid absorbent circuit, such as in liquid absorbent stream 245b and/or one or more further liquid absorbent pumps may be present.

At least a portion, preferably all of pumped spent liquid absorbent stream 215a can be passed to a spent liquid absorbent separation zone 250, in which any water and one or both of any alkali metal ions and any alkaline earth metal ions can be removed from the spent liquid absorbent via a rejection stream 255, to provide a regenerated liquid absorbent stream 245b which can be passed back to the contaminant absorption zone 200. The rejection stream 255 may comprise one or more of water, alkali metal ion and alkaline earth metal ion. The regenerated liquid absorbent stream 245b can comprise an ethylene glycol. The regenerated liquid absorbent stream 245b can be depleted in one or more of water, alkali metal ion and/or alkaline earth metal ion compared to the spent liquid absorbent stream 215.

The spent absorbent separation zone 250 may be, for instance, a distillation column or a water absorption bed. Alternatively, the spent absorbent separation zone 350 may separate the components of the pumped spent liquid absorbent stream 215a by pervaporation using a polymer membrane such as a supported polyvinyl alcohol membrane or by temperature swing absorption. Ethylene glycols are particular suitable liquid absorbents because they have boiling points of greater than 197° C. at 1 atmosphere, facilitating their separation from any absorbed water.

In the embodiment of FIG. 2, the treated regeneration gas stream 205 can be passed to a heat exchanger 260, such as a heater, where its temperature can be increased to provide treated regeneration gas stream 205a as a heated stream. In this way, heated treated regeneration gas stream 205a can be used to initiate oxidative regeneration of the deactivated catalyst in regeneration zone 100. In addition, heat exchanger 260 can control the temperature of the heated treated regeneration gas stream 205a to control the rate of the catalyst regeneration. Consequently, reducing the temperature of the heated treated regeneration gas stream 205a to a temperature below that sufficient to sustain oxidative regeneration can terminate the regeneration process.

In an alternative embodiment not shown in FIG. 2, the heat exchanger 260 may be placed downstream of the contaminant absorption zone 200, for instance to regeneration gas stream 185 or pressurised regeneration gas stream 165. However, these configurations are less preferred because of heat losses from the heated regeneration gas to the liquid absorbent that may occur in the contaminant absorption zone 200. In addition, temperature of the regeneration gas stream 185 passed to the contaminant absorption zone 200 does not require to be manipulated to control the regeneration reaction, allowing the absorption zone 200 to operate at a wider range of temperatures, particularly temperatures lower than that required to support regeneration.

Turning to the reaction zone 50, reaction effluent stream 55 comprising unreacted oxygenate, olefinic product and water can be processed in a variety of ways known in the art.

The olefinic product obtained from an OTO process can comprise ethylene and/or propylene, which may be separated from the remainder of the components in the reaction effluent stream 55. For instance, the reaction effluent stream 55 can be passed to quench zone 60, where it is separated into a water rich stream 75 comprising oxygenate and a water depleted effluent stream 65 comprising olefinic product. The separation in the quench zone 60 can be effected by contacting the reaction effluent stream 55 with an aqueous quench stream (not shown), such as a water stream, particularly a cooled water stream.

The water depleted effluent stream 65 can then be passed to an effluent compressor 70, in which the pressure of the stream is increased to provide a compressed effluent stream 85 comprising olefinic product. The effluent compressor 70 may be driven by an effluent compressor driver 80, such as an electric motor or a turbine, particularly a steam turbine. The compressed effluent stream 85 may be provided at a pressure above 5 bara, typically above 25 bara, more typically in the range of from 30 to 40 bara.

In an embodiment not shown in FIG. 2, the water depleted effluent stream 65 may be treated to remove any carbon dioxide present, for instance, prior to compression or in between compression stages if multiple stages of compression as used. For instance, the water depleted effluent stream 65 may be contacted with an aqueous alkaline stream, such as a stream comprising an alkali metal hydroxide to absorb the acid-forming gas.

Gas-liquid separators (not shown), such as a knock-out drums, for the removal of any condensed phase such as water and C5+ hydrocarbons may be present after compression, or after each stage of compression if a multi-stage compression system is used. Subsequently, the compressed effluent stream 85 can be passed to an olefinic separation zone 90, such as a distillation zone, preferably a cryogenic distillation zone, to provide two or more olefinic component streams 95, 105, 135.

The olefinic product preferably comprises two or more of the group selected from ethylene, propylene, butylene(s), pentylene(s) and hexylene(s). Consequently, each of the two or more olefinic component stream may comprise at least one of the group selected from ethylene, propylene, butylene(s), pentylene(s) and hexylene(s). In the embodiment of FIG. 2, olefinic separation zone 90 may comprise a deethaniser providing a first olefinic component stream 95 comprising ethylene, a depropaniser providing a second olefinic component stream 105 comprising propylene and a third olefinic component stream 135 comprising C4+ olefins such as one or more of butylene(s), pentylene(s) and hexylene(s).

In a preferred embodiment, at least a portion of at least one of the two or more olefinic component streams, such as the third olefinic component stream 135 comprising C4+ olefins can be passed to the OTO reaction zone 50 as olefinic co-feed stream 15 discussed above.

Where the olefinic product comprises ethylene, at least part of the ethylene may be further converted into at least one of polyethylene, mono-ethylene-glycol, ethylbenzene and styrene monomer. Where the olefinic product comprises propylene, at least part of the propylene may be further converted into at least one of polypropylene and propylene oxide.

The person skilled in the art will understand that the present invention can be carried out in many various ways without departing from the scope of the appended claims. For instance, the reaction zone and the regeneration zone may be in the same vessel, or may be one and the same zone.

1. A process for the oxidative regeneration of a deactivated catalyst comprising molecular sieve to provide a regenerated catalyst comprising regenerated molecular sieve, wherein said deactivated catalyst is obtained from one or both of an oxygenate to olefin process and an olefin cracking process, said regeneration process comprising at least the steps of:
providing a regeneration gas stream comprising oxidant;
treating the regeneration gas stream with a liquid adsorbent stream comprising an ethylene glycol in a contaminant absorption zone to remove at least a part of one or more of any water, any alkali metal ion and any alkaline earth metal ion present in the regeneration gas stream to provide a treated regeneration gas stream comprising oxidant;
regenerating a deactivated catalyst comprising molecular sieve with the treated regeneration gas stream to provide a regenerated catalyst comprising regenerated molecular sieve.

2. The process of claim 1 wherein the ethylene glycol comprises one or more of mono ethylene glycol and ethylene glycol oligomer, typically mono ethylene glycol.

3. The process of claim 1 wherein the regeneration gas stream further comprises water and one or both of alkali metal ion and alkaline earth metal ion, the treated regeneration gas stream is a water, and one or both of an alkali metal ion and alkaline earth metal ion depleted stream and the step of treating the regeneration gas stream further provides a spent liquid absorbent stream comprising an ethylene glycol, water and one or both of alkali metal ion and alkaline earth metal ion.

4. The process of claim 3, further comprising the step of:
passing at least a portion of the spent liquid absorbent stream to the contaminant absorption zone as the liquid absorbent stream.

5. The process of claim 3, further comprising the steps of:
removing a portion of the spent liquid absorbent stream as a spent liquid absorbent bleed stream;
passing an ethylene glycol to the liquid absorbent stream as a liquid absorbent restoration stream.

6. The process of claim 3, further comprising the steps of:
drying the spent liquid absorbent stream to provide a regenerated liquid absorbent stream comprising an ethylene glycol and a rejection stream comprising water and one or both of alkali metal ion and alkaline earth metal ion;
passing the regenerated liquid absorbent stream to the contaminant absorption zone as the liquid absorbent stream.

7. The process of claim 1, further comprising, between the treating of the regeneration gas stream and the regeneration of the catalyst steps, the step of:
demisting the treated regeneration gas stream to remove at least a portion of any entrained ethylene glycol.

8. The process of claim 1 further comprising, prior to passing the regeneration gas stream to the contaminant absorption zone, the step of:
heating the regeneration gas stream to provide a heated regeneration gas stream;
such that the treated regeneration gas stream is also a heated stream.

9. The process of claim 1 further comprising, between the step of passing the regeneration gas stream to the contaminant absorption zone and the step of regenerating the deactivated catalyst, the step of:
heating the treated regeneration gas stream.

10. The process of claim 1 wherein the oxidant in the regeneration gas stream comprises oxygen.

11. The process of claim 1 wherein the molecular sieve is selected from the group comprising silicoaluminophosphate and aluminosilicate.

12. The process of claim 11 wherein the molecular sieve comprises one or more of the group comprising a TON-type aluminosilicate, such as ZSM-22, a MTT-type aluminosilicate, such as ZSM-23, and a MFI-type aluminosilicate, such as ZSM-5.

13. A process for the preparation of olefinic product, the process comprising at least the steps of:
(a) reacting an oxygenate feedstock comprising oxygenate in an oxygenate to olefin reaction zone in the presence of a catalyst comprising molecular sieve to produce a deactivated catalyst comprising molecular sieve and a reaction effluent stream comprising unreacted oxygenate, olefin and water;
(b) regenerating the deactivated catalyst according to a process of claim 1 to provide a regenerated catalyst.

14. A process for the preparation of olefinic product comprising one or both of ethylene and propylene, the process comprising at least the steps of:
(a) reacting an C4+ hydrocarbon feedstock comprising olefin in an olefin cracking process reaction zone in the presence of a catalyst comprising molecular sieve to produce a deactivated catalyst comprising molecular sieve and a reaction effluent stream comprising unreacted C4+ hydrocarbon and one or both of ethylene and propylene;
(b) regenerating the deactivated catalyst according to a process of claim 1 to provide a regenerated catalyst.

15. The process of claim 13 further comprising the steps of:
(c) repeating step (a) at least once with the regenerated catalyst;
(d) repeating step (b) at least once with the deactivated catalyst.

16. An apparatus for the oxidative regeneration of a deactivated catalyst comprising molecular sieve to provide a regenerated catalyst comprising regenerated molecular sieve, said apparatus comprising at least:
a contaminant absorption zone comprising a liquid absorbent, said liquid absorbent comprising an ethylene glycol, said contaminant absorption zone having a first inlet for a regeneration gas stream comprising oxidant and a first outlet for a treated regeneration gas stream comprising oxidant, said first outlet in fluid communication with the first inlet of a regeneration zone, said contaminant absorption zone further comprising a second inlet for a liquid absorption stream and a second outlet for a spent liquid absorption stream;
a regeneration zone comprising a deactivated catalyst comprising molecular sieve, said deactivated catalyst from one or both of an oxygenate to olefin process and a olefin cracking process, said regeneration zone having a first inlet for the treated regeneration gas stream and a first outlet for a regeneration effluent stream.
